(19) United States
(12) Patent Application Publication
(10) Pub. No.: US 2024/0412886 A1
(43) Pub. Date: Dec. 12, 2024

(54) Liquid Deposition of Salts for Bombardment Target Preparation

(71) Applicant: Northstar Medical Technologies, LLC, Beloit, WI (US)

(72) Inventors: Michael KAUL, Beloit, WI (US); Dominik FRITZ, Beloit, WI (US); Jakob BAUMEISTER, Beloit, WI (US); Brandon JACKSON, Beloit, WI (US)

(73) Assignee: Northstar Medical Technologies, LLC, Beloit, WI (US)

(21) Appl. No.: 18/737,857

(22) Filed: Jun. 7, 2024

Related U.S. Application Data
(60) Provisional application No. 63/471,544, filed on Jun. 7, 2023.

(51) Int. Cl. G21G 1/10

(52) U.S. Cl. CPC G21G 1/10

(57) ABSTRACT

A target for heavy ion or electron bombardment is contemplated as is a method of its preparation. In that method, a) liquid target solution of a target metal salt dissolved in water is deposited into the bottom of a metal target capsule having an open top, and b) heated within the target capsule to form a crystalline material. Steps a) and b) are repeated until the crystalline material contains a target amount of target salt. The target capsule is then heated until the weight of the target capsule remains constant. To mitigate the formation of salt creep during the preparation, the liquid target solution can contain about 2 to about 20 percent v/v of a C₂-C₄ polyol, or the inside surface of the target capsule is coated with a hydrophobic film that provides a contact angle with water of about 70 to about 1300 prior to liquid deposition.

### CROSS-REFERENCE TO RELATED APPLICATION

This application claims priority to US application Ser. No. 63/471,544, filed on Jun. 7, 2023, whose disclosures are incorporated herein by reference.

### TECHNICAL FIELD

The present invention relates to the preparation of metal salt deposits such as those of radium, calcium, strontium, barium, zinc, copper and iron for use as targets or surrogate targets for transmutation into salts of different metals after electron beam or heavy ion beam irradiation, and subsequent use of the transmuted salt materials in medical procedures.

### BACKGROUND ART

The preparation of a radium-containing target for heavy ion bombardment in the preparation of actinium is disclosed and claimed in U.S. Pat. No. 8,349,391 to Harfensteller et al. That patent teaches the production of at least one radium-containing material dried out of an aqueous-organic solution or suspension of such a material that is applied to the surface of a hollow metallic cylindrical vessel by means of a dispersing device on a surface in such a way that the dispersing device and the surface are moving relatively towards each other. The vessel comprises a base having an outer edge that has a groove-shaped recess at the outer edge of its surface. Further, the aqueous-organic radium-depositing solution can contain an alcohol selected from the group consisting of linear and branched C₁ to C₅ alkyl alcohols; ethanol, propanol-1, propanol-2, acetone (not an alcohol) as well as mixtures thereof (column 9, lines 14-18).

The recited C₂ and C₃ alcohols and acetone have relatively low flash points and therefore their use has the potential for an explosive result that would scatter radioactive radium. For example, the flash point of ethanol is 14° C.; that of propanol-1 is 15° C.; that of propanol-2 is 12° C.; and that of acetone is −17° C.

The French scientist Wilfrid Sebaoun in 1956 published a paper [Sebaoun, Ann. Phys. (Paris), 1956, 13, 680-718], in which he described a method for preparing thin films of RaCl₂ for use in the determination of the specific activity of radium. In that paper, Sebaoun followed a “delicate” procedure described by Hufford et al. in Transuranium Elements II, Seaborg et al., McGraw-Hill, New York, page 1149 (1949). Sebaoun disclosed preparing those thin RaCl₂ films using a solution of tetraethylene glycol (TEG; HO(CH₂CH₂O)₄H) and water.

Using Ba(NO₃)₂ and BaCl₂ non-radioactive surrogate materials for radium nitrate and radium chloride, respectively, the present inventors could not reproduce the Sebaoun techniques using radium chloride using roughly 150 mg of material loaded into a target capsule using a variety of heating and layering techniques. Our work had previously shown that it is not possible to reliably deposit BaCl₂ without the use of an additive due to the aggressive nature of the salt creep as seen in FIG. 1A. For small deposits such as those of less than 150 mg, barium nitrate deposition did not require the presence of an additive such as a glycol, but the surface of the deposit was concave and non-ideal.

Sebaoun reported that their success with RaCl₂ was due to a twofold heating process. First, the solvent (water) was evaporated via infrared (IR) radiation, leaving a mixture of the salt and TEG. They then reported that the heat is increased to initiate a “partial polymerization” of the TEG that precipitates a thin film of RaCl₂, followed by the removal of the organic layer. However, that claim of “partial polymerization” was not backed by any sort of justification or evidence.

Because TEG was reported to be a successful additive for depositing radium, it was used as an additive to reduce the observed salt creep. The results of that study resembled the negative results shown in FIG. 1B in that uneven salt formation occurred and a uniform layer was not achieved. FIG. 1B illustrates the “best result” we obtained as to salt creep resulting from an aqueous BaCl₂ solution using TEG as an additive. This result was also non-ideal, providing poor results, and was difficult to repeat.

Sebaoun's method and process were targeted for thin films. Those authors reported using 0.13 to 0.17 mg/cm2 on a target having a radius of 6 mm. [Sebaoun at page 686.] The inventors' results indicated that TEG and Sebaoun's method were not suitable for thick films contemplated here and discussed hereinafter. More particularly, the amount of radium to be deposited in a target here is an about 100- to about 1000-fold increase compared to a target of Sebaoun. This doubted polymerization and our failure to reproduce Sebaoun's results led us to the present invention that is described in more detail below.

### BRIEF SUMMARY OF THE INVENTION

The present invention is broadly directed to the transmutation of one isotope or element to another by direct particle (e.g. electron or heavy ion) bombardment (or irradiation) of the first isotope or element to form a second isotope or element. These transmutations are illustrated using radium-226 as the first isotope that forms radium-225 after bombardment as the second isotope. The radium-225 spontaneously decays to form actinium-225 that is useful in medical applications.

One embodiment of this invention contemplates an aqueous target solution to be dried for the preparation of a metal salt target of a first isotope for heavy ion or electron bombardment and transmutation into a second isotope or a surrogate metal salt for the first isotope. As used herein, the phrase “heavy ion” is a charged particle having a molecular mass of one to 238 atomic mass units (AMU). That target solution comprises water containing a dissolved target metal salt or a surrogate metal salt for target metal salt present at a concentration of about 25 to about 100, and preferably at about 50 to about 75, percent by weight of solubility of that metal salt in water at room temperature. The improvement to that aqueous target solution is the presence of a dissolved C₂-C₄ polyol at about 2 to about 20 percent v/v of the solution. The C₂-C₄ polyol is removable on drying of the target solution, and its presence mitigates salt creep during drying of the target solution.

Another embodiment of the invention contemplates a method of preparation of a target for heavy ion or electron bombardment. This method comprises the steps of depositing a predetermined first amount of a liquid aqueous target solution containing a dissolved target salt present at a concentration of about 25 to about 100 percent and preferably at about 50 to about 75 percent by weight of the solubility of that target salt in water at room temperature into a metallic target capsule having an open top.

In one aspect of this embodiment, the aqueous liquid target solution also contains a dissolved C₂-C₄ polyol present at about 2 to about 20 percent, and preferably about 5 to about 10 percent, v/v of the solution, using ethylene glycol as the C₂-C₄ polyol. Other C₂-C₄ polyols are used in proportion to their molecular weights and the volumes that produce those weights. The C₂-C₄ polyol is removable on subsequent drying of the target solution, and in a preferred embodiment, the C₂-C₄ polyol has a boiling point at one atmosphere of pressure of about 300° C. or less.

In another aspect of this embodiment, the aqueous liquid target solution is deposited into a target capsule whose wall(s) are coated with a hydrophobic coating to about the level of the top of the deposited first amount of an aqueous liquid target solution, while inside bottom of the target capsule is substantially free of the hydrophobic coating. That hydrophobic coating after application and drying, and in a flat horizontal position on the metal of the target capsule exhibits a water contact angle about 70 to about 130° and decomposes at a temperature of about 225° C.

Regardless of which of the above aspects is followed, the target capsule containing the deposited aqueous liquid target solution is heated at a temperature of about 40 to about 100° C., and preferably at about 55 to about 75° C., for about 2 to about 6 hours to form a crystalline material. The depositing and heating are repeated until the formed crystalline material contains a desired target amount of target salt, while compacting the layer of crystalline material (target salt). The target capsule containing the desired target amount of crystalline material is heated with a secondary heating source at a temperature of about 200 to about 300° C. for a time period of about 4 hours or until the weight of the target capsule when cooled to about room temperature (e.g., about 20 to about 25° C.) remains constant within a standard deviation of about 1 to about 5%. Such a constant weight should match the theoretical target mass (i.e., the mass after waters of crystallization, C₂-C₄ polyols and hydrophobic material, when present, have been removed to form the target). Exemplary secondary heating devices include but not limited to: a small oven, muffle furnace, powerful IR bulb, low power tube heater, and a hot plate that gradually heats the target capsule at a temperature of about 240 to about 300° C. for a time period of about 4 hours

Illustrative radioactive target salts include RaCl₂, RaBr₂ and Ra(NO₃)₂, whereas BaCl₂, BaBr₂ and Ba(NO₃)₂ are used as water-soluble, non-radioactive surrogate targets for the radioactive salts. Further target salts and surrogates are discussed hereinafter.

In preferred aspects of the above embodiment, the about room temperature target capsule containing a desired amount of target metal salt is sealed; i.e., the open top of the target capsule is sealed closed. In another preferred aspect, the first heating of step lasts for about 2 to about 3 hours. In another preferred aspect, subsequent to that first heating step, subsequent heating steps last for about 4 to about 6 hours. In a still further aspect of the above embodiment, the C₂-C₄ polyol is ethylene glycol.

A second embodiment of the present invention contemplates an improved aqueous target solution for the preparation of a target for heavy ion or electron bombardment that comprises water containing a dissolved target salt present at a concentration of about 25 to about 100 percent, and preferably at about 50 to about 75 percent, by weight of the solubility of that salt in water at room temperature, the improvement that is the presence of a dissolved C₂-C₄ polyol present at about 2 to about 20 percent, and preferably about 5 to about 10 percent, v/v of the solution using ethylene glycol (EG) as a C₂-C₄ polyol. Other C₂-C₄ polyols are used in proportion to their molecular weights and the volumes to EG that produce those weights. The water used herein is distilled or deionized water.

It is to be understood that ethylene glycol is the preferred C₂-C₄ polyol, and that it is preferably used as the only C₂-C₄ polyol present in the target solution. However, one or more of the C₂-C₄ polyols can be used in combination, with the boiling points of each of the polyols used appropriate for the capsule used as is the temperature of the one or more final heatings so that the integrity of the capsule is not impaired.

In one preferred aspect of this embodiment, the C₂-C₄ polyol is ethylene glycol. In another preferred aspect, the dissolved target salt is radium chloride, radium bromide or radium nitrate. In a third preferred aspect of this embodiment, the aqueous target solution contains only water, distilled or deionized, the dissolved target salt and the dissolved C₂-C₄ polyol. A third preferred aspect of the invention is the use of a hydrophobic coating on the inner vertical surface of the capsule instead of the C₂-C₄ polyol to inhibit salt creep.

Another embodiment of present invention is directed to a capsule for liquid deposition fabrication of a target for heavy ion or electron bombardment.

Yet another embodiment of present invention is directed to a capsule for liquid deposition fabrication of a target for heavy ion or electron bombardment wherein an interior wall surface, but not the bottom of the capsule is coated with a hydrophobic coating. That coating is typically removed after successful deposition.

A still further embodiment of the present invention is directed to a capsule for liquid deposition of a target for heavy ion or electron bombardment wherein the capsule includes one or more ridges extending from an interior bottom of the capsule.

In one aspect of this embodiment, the ridges are formed in a circular shape on the bottom of the capsule.

In another aspect of this embodiment, the ridges are coated with a hydrophobic coating.

In another aspect of the present invention, one or more of the above embodiments are utilized together to fabricate the target.

### BRIEF DESCRIPTION OF THE DRAWINGS

In the drawings forming a portion of this description,

FIG. 1, in two panels as FIGS. 1A and 1B, are (FIG. 1A) a photograph of a target capsule in which an aqueous solution of barium chloride was dried under an infrared lamp heater showing unacceptable salt creep during bulk target fabrication, and (FIG. 1B) a photograph that shows a “best result” obtained as to salt creep resulting from an aqueous BaCl₂ solution using 10% by volume tetraethylene glycol

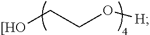

TEG] as an additive where the creep is shown at least on the upper surface of the inner portion of the capsule, with a heating profile of 65° C. for 2 hours and a furnace at 250° C. for 4 hours (hereinafter, “standard conditions”);

FIG. 2, in two panels as FIGS. 2A and 2B, are (FIG. 2A) a photograph of a target capsule in which an aqueous solution of the same concentration of barium chloride as in FIG. 1 plus ethylene glycol in accordance with this invention using standard conditions for drying of FIG. 1 and showing little if any salt creep, and (FIG. 2B) a photograph showing the appearance of the petrolatum-like appearance of a deposited layer after an initial heating/drying as in FIG. 1;

FIG. 3 illustrates a device for fabrication by liquid deposition of a target by deposit of the final target solution into a target capsule and heating of the capsule;

FIG. 4 illustrates a further embodiment of the device of FIG. 3 with a plate in thermal contact with the underside of the target capsule;

FIG. 5 is an illustrative scheme showing the steps in fabrication of a target fabricated in accordance with this invention;

FIGS. 6A and 6B, illustrate a target capsule with a hydrophobic coating applied thereon;

FIG. 7 illustrates a target capsule with specially fabricated geometrical features; and

FIG. 8 illustrates a cross-sectional view of the target capsule of FIG. 6 with circular ridges.

### DETAILED DESCRIPTION OF PREFERRED EMBODIMENTS

Although this invention is susceptible of embodiments in many different forms, embodiments that are shown in the drawings and will be described in detail herein in specific embodiments with the understanding that the present disclosure is an exemplification of the principles of the invention. It is not intended to limit the invention to the specific illustrated embodiments. The features of the invention disclosed herein in the description, drawings, and claims can be significant, both individually and in any desired combinations, for the operation of the invention in its various embodiments. Features from one embodiment can be used in other embodiments of the invention.

The present invention is directed to the transmutation of one isotope or element to another by direct particle (e.g. electron or heavy ion) irradiation of the first target isotope or element to form a second product isotope or element. Several potential target metal salts have been examined to determine their possible use as targets for such transmutation.

These potential target metal salts have been deposited from an aqueous solution of the proposed salt into a capsule or container used to contain the target material during irradiation only to have target preparation thwarted by the growth of salt crystals up the internal walls of the capsule during the drying process of the aqueous solutions. Such salt crystal growth is referred to herein as “salt creep”.

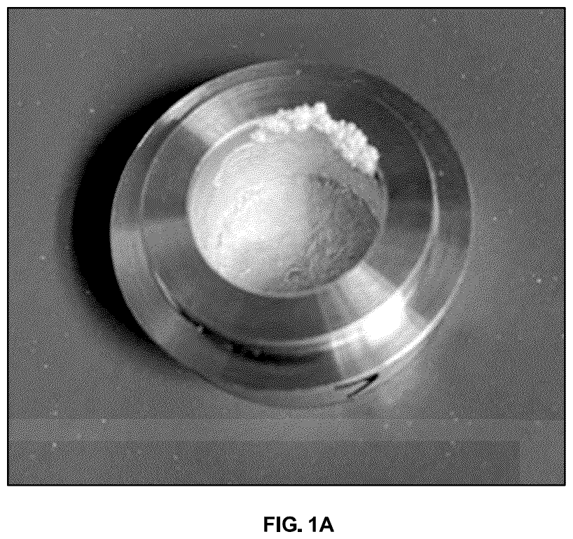

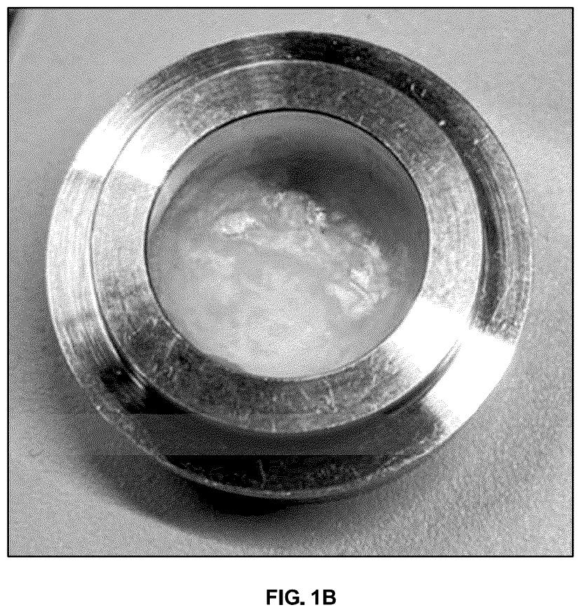

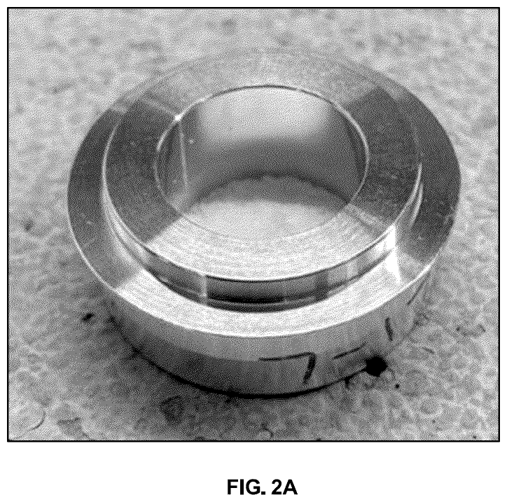

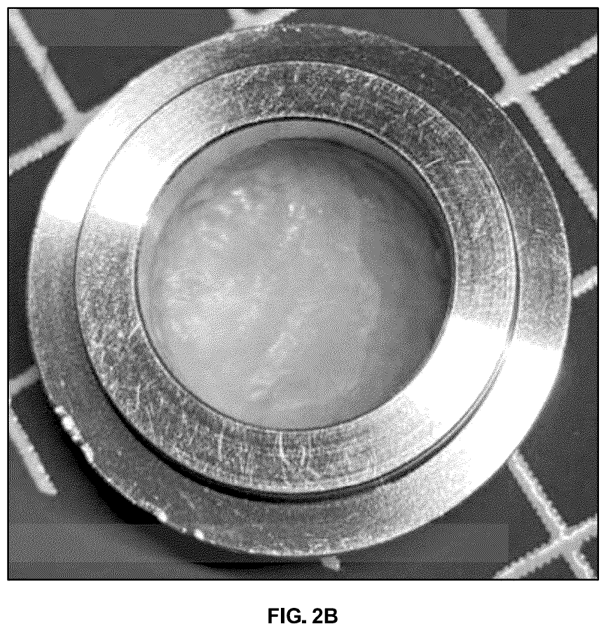

Typical salt creep is shown in FIG. 1A. FIG. 1B illustrates significant salt creep using a material (tetraethylene glycol) suggested by the prior art as useful to overcome salt creep but as seen in that Figure, in FIG. 1B was not particularly useful. FIG. 2A illustrates the result of using the present invention to mitigate salt creep as is described hereinafter.

Salt creep causes the target material to be more spread out in the target container, thereby lessening the effect of the irradiation because the particle beams are constrained to a narrow cross-section for efficacy and safety. In addition, the salt creep lessens the density of the target material that also leads to a lower yield of transmuted product isotope.

An important aspect of the present invention is to mitigate (lessen or inhibit) salt creep so that the prepared target remains at the bottom of the target capsule with little or no creep up the capsule walls. Each of the metal salts that can be used as first isotopes or surrogates for first isotopes discussed in the Examples has been found to respond well to a salt creep mitigation procedure discussed therein to provide dried targets similar to that shown in FIG. 2A.

The target compounds to be transmuted in electron beam targets and those used in heavy ion beam targets can be prepared similarly to each other. Such targets contain an isotope that is intended to be transmuted by irradiation by an electron beam or a heavy ion beam into a second isotope, or the target can contain a surrogate isotope for the target isotope that is non-radioactive of another element and exhibits similar chemical and water-solubility of at least some salts formed by both elements.

Illustrative targets for electron beam irradiation include water-soluble salts of radium-226 such as RaCl₂, RaBr₂ or Ra(NO₃)₂ that can be used to produce actinium-225. Non-radioactive stand-in (surrogates) for those radium salts include BaCl₂, BaBr₂ or Ba(NO₃)₂. For ease in explanation and limit verbosity, the above radium salts and barium salt surrogates are used to represent the other target salts and surrogates.

Similarly, electron beam irradiation leading to photoneutron production using a water-soluble salt of fluoride-19, such as potassium or sodium fluoride can be used as a target for production of fluoride-18 and a similar salt of bromine-79 can be used to form the salt of bromine-77; calcium-48 chloride or bromide can be used to prepare scandium-47 chloride or bromide; a water-soluble salt of copper-65 or zinc-66 such as the chloride or bromide can be used to make copper-64; a chloride or bromide of gadolinium-69 can be used to form the same salt of gadolinium-67; a potassium or sodium salt of a molybdate prepared from molybdenum-100 can be used to form the same salt of molybdenum-99.

On the other hand, electron beam irradiation leading to photoproton production using a water soluble salt of calcium-44 can be used to form the same salt of potassium-43; a tri- or tetrachloro salt of titanium-48 can be used to produce a chloride salt of scandium-47; chloride or bromide salt of nickel-58 can be used to form a corresponding salt of cobolt-57; a chloride or nitrate salt of zinc-68 can be used to form the same salt of copper-67; zirconium-91 nitrate can be used to form yittrium-90 nitrate; barium-131 chloride can be used to form cesium-131 chloride; and erbium-167 chloride or bromide can be used to form the corresponding chloride or bromide of holmium-166.

Illustrative targets for heavy ion beam irradiation include the chloride or bromide of zinc-69 that can be used to form the same salt of gadolinium-68; the chloride, bromide or nitrate of rubidium-85 can be used to prepare the corresponding salt of strontium-82; the chloride or bromide salt of nickel-64 can be used to form a corresponding salt of copper-64; strontium-86 nitrate can be used to prepare yittrium-90 nitrate; and ytterbium-176 chloride or bromide can be used to form the corresponding lutetium-177 salt.

Illustrative target first isotopes and their surrogates are listed in the table below. In that table, the target isotope is listed with a whole number molecular weight whereas the surrogate isotope is listed with a rational number molecular weight as it is a weighted average of the molecular weights of the isotopes of the individual elements.

Illustrative Transmutation Target First Isotopes and Surrogates

| | | | |
|---|---|---|---|
| fluorine-19 | calcium-44 | calcium-48 | calcium-40.1 |
| bromine-79 | zinc-66 | zinc-68 | zinc-65.4 |
| gadolinium-69 | molybdenum-100 | calcium-44 | titanium-48 |
| nickel-58 | nickel-64 | zirconium-91 | erbium-167 |
| barium-131 | barium-137.3 | rubidium-85 | ytterbium-176 |
| strontium-86 | strontium-87.6 | copper-65 | copper-63.5 |
| iron-55.8 | radium-226 | — | — |

It is to be understood that a target first isotope need not be present in the target capsule as an isotopically pure metal salt. Rather, the target isotope in this invention can be present in a target metal salt mixture of parental isotopes (those from which the target isotope was formed), or in a mixture of surrogate metal salts.

As used herein, a “surrogate” isotope is a non-radioactive isotope of a first isotope or a non-radioactive isotope of another element exhibiting chemistry and water-solubility of at least some of its salts that is similar to those of the radioactive target isotope for which it is a surrogate. For example, zinc-65.4 can be a surrogate for zinc-66 or zinc-68. Similarly, calcium-40.1 can be a surrogate for calcium-44 or calcium-48. Radium has no known non-radioactive isotopes, but lighter-molecular weight barium is just below it in the column of alkaline earth elements of the Group 2 column of the Periodic Table and exhibits similar chemistry as does the even lighter molecular weight element strontium. In addition, barium and radium dichlorides form isomorphic dihydrate crystals.

It is to be understood that each of the target isotopes or their surrogates and the products made by their irradiation noted above are well-known in the art for being precursors and desired products useful in one or more medical procedures. Similarly, methods of preparing the target isotopes and separating target and product isotopes after irradiation are well-known to workers skilled in this art and need not be discussed herein for that reason.

In an exemplary embodiment, a volume of a solution containing distilled or deionized water and a predetermined amount of a C₂-C₄ polyol, such as ethylene glycol (EG), glycerin, a propylene glycol (PG) or other C₂-C₄ polyol, is mixed with a predetermined amount of a target salt sufficient to deposit a desired salt target mass, such as a RaCl₂, RaBr₂ or Ra(NO₃)₂, respectively, and the composition so made is homogenized to form a final target solution. A predetermined amount of that final target solution, such as about one-half, is dispensed into a target capsule and an infrared (IR) lamp is placed above the target capsule to heat the deposited final target solution and begin the process of evaporating the solvent, mostly water, out of the deposited solution.

The contemplated mass of radium for deposition into the target capsule is about 10 mg to about 1 gram. More than 1 gram can also be used, but the amount stated has been found to be comfortable for manufacture and handling of a contemplated nuclide. Preferred amounts are about 10 mg to about 750 mg, and more preferably about 100 to about 500 mg. The tables below show the weight of radium or barium to be deposited in the left columns and the mass of the three salts of each element that are deposited to achieve the desired amount of each of those elements.

| (Target) | | | |
|---|---|---|---|
| Mass Of Radium (g) | Mass of RaCl₂ | Mass of RaBr₂ | Mass of Ra(NO₃)₂ |
| 0.0100 | 0.0131 | 0.0171 | 0.0155 |
| 0.1000 | 0.1314 | 0.1707 | 0.1549 |
| 0.2500 | 0.3284 | 0.4268 | 0.3872 |
| 0.5000 | 0.6569 | 0.8536 | 0.7744 |
| 0.7500 | 0.9853 | 1.2803 | 1.1615 |
| 1.0000 | 1.3137 | 1.7071 | 1.5487 |

| (Target) | | | |
|---|---|---|---|
| Mass of Barium (g) | Mass of BaCl₂ | Mass of BaBr₂ | Mass of Ba(NO₃)₂ |
| 0.0100 | 0.0152 | 0.0216 | 0.0190 |
| 0.1000 | 0.1516 | 0.2164 | 0.1903 |
| 0.2500 | 0.3791 | 0.5409 | 0.4758 |
| 0.5000 | 0.7581 | 1.0819 | 0.9515 |
| 0.7500 | 1.1372 | 1.6228 | 1.4273 |
| 1.0000 | 1.5163 | 2.1637 | 1.9030 |

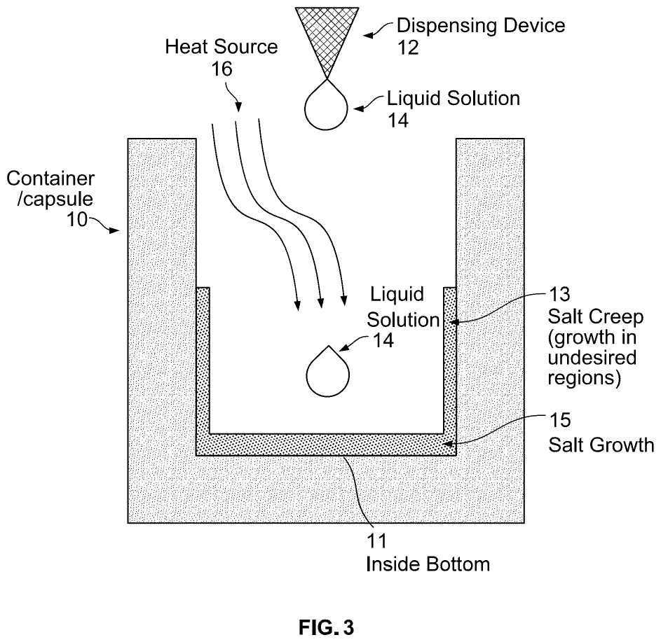

FIG. 3 schematically illustrates a device for fabrication by liquid deposition of a target by deposit of the final target solution into a target capsule and heating of the capsule 10.

In an exemplary embodiment, the device of FIG. 3 includes a capsule (container) 10. The capsule 10 is a target capsule into which dispensing device 12 that dispenses an aqueous liquid solution 14 containing the radium salt, another salt to be irradiated, or a surrogate for use in in learning how to carry out target preparation. Capsule 10 holds the target solution at varying concentrations at different times during the target deposition process.

The target capsule 10 can be made of one or more of aluminum, steel, silicon carbide, copper, tantalum or tungsten. In one embodiment, the target capsule is metal, preferably an aluminum capsule 10. In a further preferred embodiment, the material of the capsule is aluminum Al-1050. In an alternative embodiment, the capsule is machined from aluminum Al-6061. In a further embodiment, the capsule is sealed, preferably by welding, after deposition, heating and drying of the target solution.

A dispensing device 12 contains the final target solution 14, such as the target solution described above. The dispensing device 12 dispenses the target solution 14 into target capsule 10. This can be by a drip or a larger volume, such as a stream. An automated syringe pump or peristaltic pump is used in production. With proper shielding in place, a hand syringe can be used.

A heat source 16 is located over capsule 10. One example of a heat source is an infrared (IR) lamp that applies radiative heating to the target solution. The heat source is provided to drive off solvent, e.g., water and C₂-C₄ polyol from the target solution 14 within target capsule 10. The heat can come from many different pathways.

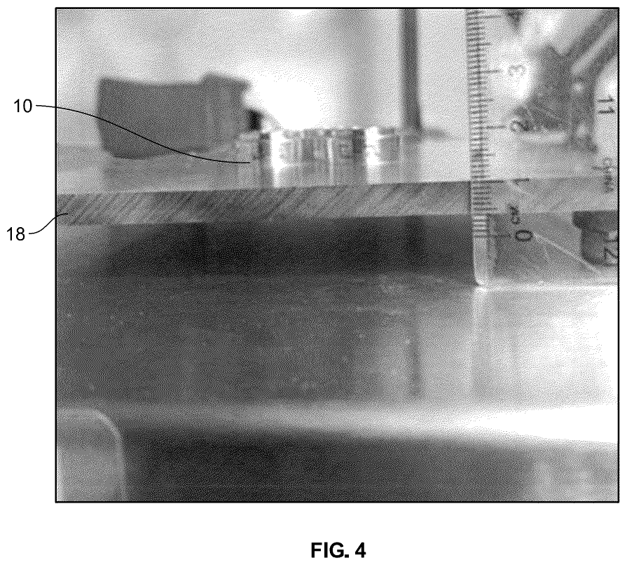

In a further embodiment, a plate 18 is provided underneath and in thermal contact with the bottom surface (e.g., the inside bottom) 11 of the target capsule 10. In an exemplary embodiment, the plate 18 is made from a material with a high thermal conductivity. We have found that a salt creep 13 can be greatly reduced by having the target capsule 10 in thermal contact with (rest upon so that heat transfers from the bottom of the capsule to) a plate 18 (FIG. 4) that is itself preferably cooled from the bottom (illustratively, using natural convection with air). The plate 18 can be utilized with the device of FIG. 3 described above. Further, the plate 18 can be utilized with the final target solution described above.

Without wishing to be bound by theory, it is believed that the plate surface on which the underside of the capsule rests provides active cooling to the capsule 10 by serving as a heat sync for the heat deposited in the capsule/salt solution. A gap underneath the plate permits air to flow through, convectively-cooling the plate and thereby the bottom of the capsule 10.

For preferred functionality, the plate is a material with a high thermal conductivity, such as silver, copper or aluminum, to minimize the resistance to heat removal from the capsule. One preferred high thermal conductivity material for the plate is aluminum. The goal is to maximize the energy deposition directly in the salt solution (and more specifically, directly in the water of the solution), while minimizing the temperature of the surrounding surfaces.

We have discovered that when using another material with a lower thermal conductivity, such as steel or wood, the salt creep was noticeably worse. Additionally, resting a high thermal conductivity plate on a surface with low thermal conductivity served to reduce the benefit of the high thermal conductivity plate.

In a further embodiment, the bottom capsule surface of target capsule 10 that contacts the high thermal conductivity plate 18 may or may not be actively cooled, such as by forced convection. It can also be cooled conductively or convectively.

With regard to the device of FIG. 3, as the target capsule heats, water is carefully evaporated off under action of IR radiation to help minimize the amount of air pockets or bubbles that are formed. Once a majority of the water is removed, what remains is a petrolatum-like deposit; i.e., the once liquid solution becomes thickened and gel-like, resembling petroleum jelly as is shown in FIG. 2B. The target is then permitted to continue to heat under the lamp, and after several hours a white deposit remains that contains the target salt being deposited and some form of the C₂-C₄ polyol (See, FIG. 2). At this stage, the target is referred to as “dry”.

Once the first amount of target solution is “dry”, the second amount is added. In previous studies when using other or no additive; i.e., without the C₂-C₄ polyol, the initially deposited and dried target salt was re-dissolved by the addition of the second target solution, leading to increased volume in the target capsule, resulting in increased salt creep. In accordance with the present invention, on dispensing the second target solution, when first and second volumes were about of equal volume, the initial initially deposited and dried target salt material “sorbs” (absorbs or adsorbs) the second target solution and minimal volume increase is observed in the target capsule. The same heating process was followed as above and the target was thoroughly heated by the IR lamp until a very compact and uniform deposit remained.

It is currently preferred to utilize two about equal amounts of target solution and two drying steps when fabricating a target of about 100 mg or less. However, three, four, five or more additions can be utilized, providing multiple salt solution additions.

Finally, the target capsule and its contents are heated to about 200 to about 300° C., and preferably to about 240 to about 300° C. in a secondary heating device such as, but not limited to: a small oven, muffle furnace, powerful IR bulb, low power tube heater, and a hot plate that gradually heats the target capsule to a temperature of to about 200 to about 300° C., and preferably to about 240 to about 300° C. for a time period of about 4 hours to drive off any waters of crystallization, to remove C₂-C₄ polyol from the target salt, or remove the hydrophobic coating (discussed below) when present. It is believed that this multiple salt solution addition/heating process and the use of the C₂-C₄ polyol each contribute in making this technique work so successfully in minimizing salt creep.

Initial studies indicated that it is of import to heat the solution very slowly and to only use the action of the IR lamp to excite and remove the water molecules, while leaving the C₂-C₄ polyol, or hydrophobic coating, and salt-containing petrolatum-like deposit in the capsule.

Under the current set of parameters and equipment, in the first heating(s) (liquid through petrolatum stages) the deposited target solution is heated within the target capsule at a temperature of about 40 to about 100° C., and preferably at about 55 to about 75° C., for about 2 to about 6 hours to form a crystalline material. Moving away from this range of temperatures promotes the target solution to creep similarly to what is observed with just the neat salt (FIG. 1).

After heating with the IR lamp is completed, for removal of the waters of crystallization from deposited barium chloride and therefore also radium chloride, it is reported [Peter et al., IJSR 8 (8): 1775-1779 (August 2019) that a temperature of about 150 to about 180° C. is sufficient. However, a present aqueous target solution contains about 2 to about 20 percent, and preferably about 5 to about 10 percent, v/v of a C₂-C₄ polyol that has a boiling point above that of water at one atmosphere of pressure (760 mm Hg). This heating can also be carried out in a vacuum furnace or otherwise under reduced pressure using lower temperatures as is known by skilled workers.

More specifically, illustrative C₂-C₄ polyols include ethylene glycol, 1,2-propylene glycol, 1,3-propylene glycol, glycerin, 1,2-, 1,3-, 1,4-, and 2,3-butanediols, 1,2,3-trihydroxybutane, 1,2,4-trihydroxybutane, 2-methyl-1,2-propanediol, erythritol, and threitol. At one atmosphere of pressure, ethylene glycol boils at roughly 200° C. with a flash point of 111° C.; propylene glycol boils at about 187° C. with a flash point of about 99° C.; 1,3-propanediol boils at about 214° C. and has a flash point of about 140° C.; glycerin boils at about 290° C. and has a flash point of 160° C.; and erythritol boils at about 329-331° C. The 1,2-, 1,3-, 1,4-, and 2,3-butanediols, have the following boiling points at one atmosphere of pressure: 1,2-192-194° C.; 1,3-204-207° C.; 1,4-230° C.; 2,3-180-182° C., respectively. 2-Methyl-1,2-propanediol boils at 290° C. Of the C₄ polyols, C₄-diols are preferred.

As discussed before, aluminum is the presently preferred material from which the capsule 10 is made. The capsule can also be made from other materials such as steel and various steel alloys. Work hardened aluminum alloys, such as those preferably used here, tend to lose their post-annealing properties at temperatures above about 300° C. As a consequence, it is most preferred to use a C₂-C₄ polyol whose boiling point at one atmosphere of pressure is about 300° C. or less when using a preferred aluminum capsule. A final temperature of about 240 to about 300° C. was selected, using the lower temperatures for the lower boiling points and the higher temperatures for the higher boiling polyols. It is to be noted that the above C₂-C₄ polyols each have higher flash points than those mono-alcohols of Harfensteller et al., and thereby provide a safer work environment.

An exhaust fan typically positioned above the heating capsule 10 can also be included to assist in removing the evaporating gases from the immediate area of the heating capsule 10. When a hydrophobic coating rather than C₂-C₄ polyols is used to retard salt creep, heating to remove the waters of crystallization, degrade and remove the hydrophobic coating is also carried out as above at a temperature of about 200 to about 300° C., and preferably about 240 to about 300° C.

As currently practiced, the stopping point of the heating process is determined gravimetrically. When a plateau of mass loss is achieved, the heating process is complete and a comparison of the measured salt mass to theoretical mass values is performed.

During the initial heating-drying step, target solution starts as a clear, colorless solution prior to heating. Once IR heating is initiated (after about an hour), the salt-polyol deposit starts to form as the solvent is removed, and this is what looks like petroleum jelly. Finally, as the deposit is continued to heat under the IR lamp, the deposit starts to harden and look more white/crystalline in nature (after 2-3 hours) as seen in FIG. 2. Our studies indicate that the process works better if the target is permitted to dry for this total amount of time, which corresponds to the visual change in appearance observed.

In carrying out preparation of a radium/barium target, RaCl₂ has a solubility in water of 24.5 g/100 mL, with barium chloride is more soluble, with a solubility of about 35 g/100 mL. Exemplary concentrations of barium chloride and therefore radium chloride used are about 50 to about 75 percent of the solubility of each in distilled or deionized water at room temperature. Illustrative initial target solutions utilize 24 g BaCl₂/100 mL; calculating to about 68.5% of the solubility of BaCl₂. A similar radium chloride initial target solution contains about 12 to about 17 g RaCl₂/100 mL.

Current studies have shown a concentration about 2 to about 20 percent, preferably about 5 to about 10 percent, percent of the C₂-C₄ polyol provides satisfactory results. A more preferred concentration of about 5 to about 7 percent of the preferred polyol, ethylene glycol, (v/v) yields ideal results.

The inventors have determined that a hydrophobic coating on the inner wall or walls [wall(s)] of the capsule can also mitigate salt creep. Thus, in another exemplary embodiment, a hydrophobic coating 20 is applied to interior wall(s) of target capsule 10 prior to target liquid deposition. When the hydrophobic coating is used, it is preferred to exclude the C₂-C₄ polyol from the target liquid. The hydrophobic coating is a material that does not react with the target material, the liquid or salt components of the final target solution and can be removed from the capsule 10 wall(s) using the heating regimen discussed above for C₂-C₄ polyol-containing target compositions.

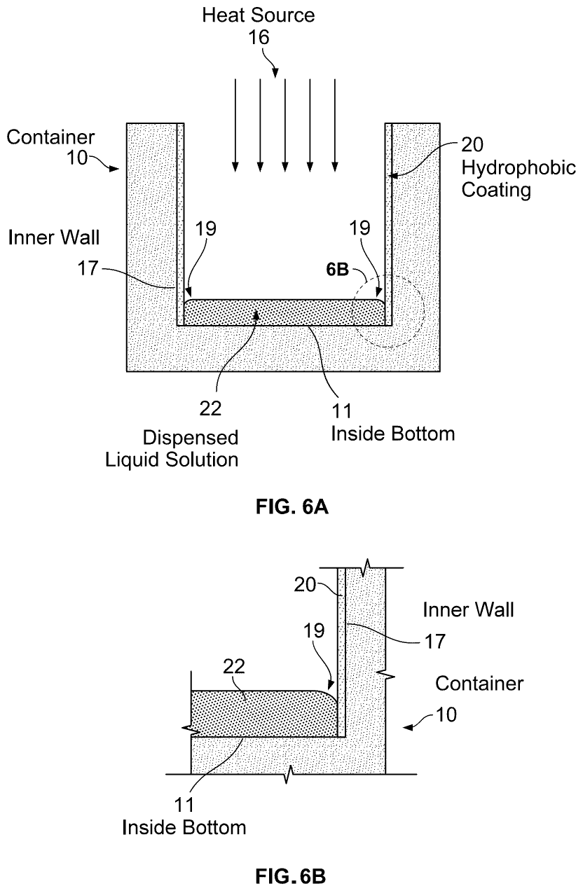

FIG. 6A illustrates an aspect of this embodiment wherein a hydrophobic coating 20 is applied to the wall(s) of capsule 10 and dried prior to dispensing the target solution. After application of the hydrophobic coating, the target solution without C₂-C₄ polyol is dispensed and accumulates on the bottom of capsule 10 as dispensed solution 22. In one embodiment, the hydrophobic coating 20 results in a thin film on the inner surface of the inner wall(s) of capsule 10. The thin film can have a thickness of greater than 1 and up to about 1000 nm, and preferably, a thickness of about 2 to about 100 nm.

A successful hydrophobic coating after application and drying, and in a horizontal position exhibits a water contact angle about 70 to about 1300, and preferably about 80 to about 120°, to mitigate salt creep. For target sizes up to 150 mg of salt material (depending on exact chemical composition), a water contact angle of at least about 90° is preferred. However, in order to extend the target masses up to 1,000 mg or greater of salt material, water contact angles of about 110° C. or greater are necessary.

It should be noted that the above masses are for a container with an internal volume of about 0.2 to about 5 cm3, with a volume presently preferred being about 1.2 cm3. Although larger containers permit more physical mass of salt to be present, the methods described herein allow for significant mitigation of salt creep and therefore efficient target compaction. This target compaction is of some import for irradiation as it results in dramatically increased production efficiencies and improved space utilization.

More specifically, the surface of the inner wall(s) 17 of capsule 10 is partially coated with the hydrophobic coating. In this aspect, the entirety of the inner wall surface is not coated with the hydrophobic coating, but instead the coating is applied up to about where the level of the top of the deposited target solution. The treating of the inner wall(s) 17 of the capsule 10 with the hydrophobic coating results in a specialized and removable surface finish to these walls. The inside bottom 11 of the container 10 is substantially free (about 90 to about 100% free) of the hydrophobic coating to permit the dried salt to adhere to the inside bottom 11 of the container (capsule) 10. After the target solution has been dispensed, liquid deposition is performed as explained previously. During liquid deposition, the hydrophobic coating produces a contact angle 19 (θ_{C}) of about 70° to about when the solution contacts the hydrophobic coating on the wall of capsule 10. This is illustrated in FIG. 6B. Use of a hydrophobic coating permits bulk target fabrication by mitigating salt creep. The hydrophobic coating produces ideal solution contact angle to mitigate salt creep.

During the final heating step of the final target solution deposition, the thin film coating of the hydrophobic coating is decomposed and vaporized with minimal residuals left behind. The plate 18 of FIG. 4 can be utilized with the target capsule 10 with hydrophobic coating noted above and discussed hereinafter.

Although the present invention is not limited to a specific material for the hydrophobic coating, illustrative examples of two hydrophobic coatings as thin film coatings are as follows:

In a first example, the inventors used Aculon® AL-B (Aculon, Inc., San Diego, CA) as a hydrophobic coating. In this example, the hydrophobic coating is applied with a thickness in the range of approximately 2-4 nm. The onset of decomposition for this hydrophobic coating occurs at approximately 225° C. Following the standard conditions for heating, the hydrophobic coating was removed from the capsule 10 by heating up to approximately 250° C. for 5 minutes to decompose polymer layer.

It is understood that the Aculon® AL-B hydrophobic coating is designed for coating a metal substrate, and is disclosed and described in U.S. Pat. No. 8,178,004, whose disclosures are hereby incorporated by reference. Broadly speaking, the hydrophobic coating contains: (a) 0.1 to 10 percent by weight of a phosphorus acid such as an organo phosphoric acid, an organo phosphinic acid or an phosphonic acid, having perfluorinated hydrocarbon groups capable of forming a self-assembled monolayer on the metal substrate; (b) 0.1 to 10 percent by weight of a surfactant that is structurally different from (a); (c) 2 to 30 percent by weight of an organic solvent; and (d) 50 to 95 percent by weight water; with the percentages by weight being based on the total weight of (a), (b), (c) and (d). Preferably, the above acid having perfluorinated hydrocarbon groups is of the structure:

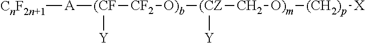

where A is an oxygen radical or a chemical bond; n is 1 to 20; Y is H, F, CₙF₂ₙ₊₁, CₙH₂ₙ₊₁, Z is H or F; b is 0 to 50; m is O to 50; p is 1 to 20; and X is a group selected from a phosphoric acid, a phosphinic acid and a phosphonic acid.

This hydrophobic coating is typically present at 1-5 percent by weight in a solvent such as a mixture of about 50 percent ethanol, about 2% of each of methanol and iso-propanol plus about 42% of a mixture of 2-(difluoromethoxymethyl)-1,1,1,2,3,3,3-heptafluoropropane and 4-methoxy-1,1,1,2,2,3,3,4,4-nonafluorobutan. The coating composition has a viscosity of about 2 cP, and when applied to a metallic surface by wiping or dipping provides a coating thickness of about 2 to about 4 nm. On a flat, horizontal target capsule metal surface, the coating provides a water contact angle of about 116°, and a sliding angle of a water droplet of about 20 to about 30°. On drying, this hydrophobic coating degrades at about 225° C.

In a second example, the inventors used Aculon® Alt #6 as a hydrophobic coating. It is understood that the Aculon® Alt #6 hydrophobic coating is disclosed and described in U.S. Pat. No. 7,879,437, whose disclosures are hereby incorporated by reference.

This coating is described as a reaction product of (a) a transition metal compound in which the transition metal is selected from niobium and transition metals such as tantalum, titanium, zirconium, lanthanum and tungsten having electrons in the f orbital and in which the transition metal compound has ligands selected from alkoxide, halide, keto acid, amine and acylate, and (b) a silicon-containing material. The silicone-containing material has a formula selected from: R¹₄₋ₓSiAₓ or (R¹₃Si)_{y}B or an organo(poly)siloxane and an organo(poly)silazane having units of the formula:

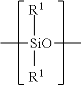

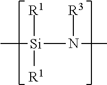

where:
   R¹ are identical or different in each presence and are a hydrocarbon or substituted hydrocarbon radical containing from 1 to 100 carbon atoms; A is hydrogen, halogen, OH, OR² or O—C(O)—R²; B is NR³_{3-Y}; R² is a hydrocarbon or substituted hydrocarbon radical containing from 1 to 12 carbon atoms; R³ is hydrogen or is the same as R¹, x is 1, 2 or 3; and y is 1 or 2.

In some preferred compositions, the silicon-containing material has the formula:
R¹₄₋ₓSiAₓ, where R¹ is a fluoro-substituted hydrocarbon and A is OR², in which R¹ is a fluoro-substituted hydrocarbon having the structure

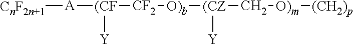

where A is an oxygen radical or a chemical bond; n is 1 to 6; and Y is F or CₙF₂ₙ₊₁; b is 1 to 10; m is O to 6 and p is O to 18. A coating of the above reaction product is dissolved or dispersed for application in an organic solvent such as isopropyl alcohol, hexamethyldisiloxane, or methylene chloride for application to the metal surface.

In this example, the hydrophobic coating is applied with a thickness in the range of about 20 to about 100 nm by dip coating. On a flat metallic surface, the coating provides a water contact angle of about 90°, and a sliding angle of a water droplet of about 30°. After application and drying on a metallic substrate such as aluminum, followed by deposition of the aqueous target or surrogate target aqueous solutions and their low temperature drying step or steps, the final drying step of heating the target-containing or surrogate-containing container to approximately 240 to about 300° C. for about 4 hours to decompose this fluoride/silicone-based layer.

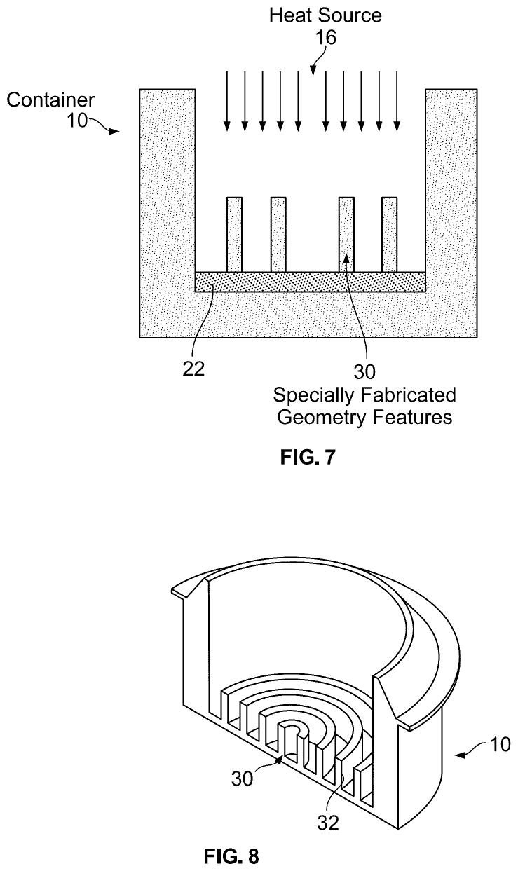

In as illustrated in FIG. 7, the capsule 10 can have specially fabricated geometrical features, such as ridges 30. The special geometrical features or ridges 30 are provided within the capsule 10 to geometrically mitigate against salt creep.

In this embodiment, in a similar manner as discussed above, the target solution is dispensed from a dispensing device 12 and accumulates on the bottom of capsule 10 as dispensed solution 22. In this embodiment, the target solution is dispensed on the bottom of the capsule 10 around the ridges 30. The ridges provide physical isolation of the target solution that results in less migration of solvent during evaporation.

In addition, the ridges significantly increase capsule 10 surface area to target solution volume ratio in the region of interest resulting in reduction of salt creep, as well as improvement to heat removal from the salt during subsequent irradiation. As a result, the dispensed target solution is subdivided into smaller components through machined geometric features. Some of the many benefits of this configuration directly stem from increased surface area for the salt to contact, increased maximum salt capacity, and increased heat removal from salt.

In one exemplary aspect of this embodiment shown in cross-section in FIG. 8, the ridges 30 are formed as a series of circular ridges 32 on the bottom of capsule 10. The present invention contemplates other possible ridges and shapes for the special geometry features 30 on the bottom of capsule 10. In this aspect, circular shapes are shown as the ridges as the inventors believe that such circular shapes are the most easily manufactured. However, other shapes (e.g., rectangular) can be used with a target capsule 10 geometry change as to a rectangle.

The present invention also contemplates various configurations and is not limited to the number of special geometry features or ridges 32. The desired distance between ridges can be dependent on manufacturability. This distance and manufacturability can also be influenced by ridge height. The goal of the configurations of the special geometry features or ridges is to maximize the surface area that the salt contacts, in accordance with manufacturability.

The number of ridges can also be dependent on ridge thickness. The thinner the thickness of the ridges, the more possible ridges that can possibly be included. The minimum distance between the ridges would have to provide enough room for the final target solution to fall into the valleys between the ridges to form the dispensed final target solution 22 on the bottom of the capsule 10 between the ridges.

It is also desirable for the target solution to not remain on top of the special geometric features or ridges due to surface tension. In light of this, it is desirable for the thickness of the ridges or the circles to be as thin as possible within manufacturing limits. Thus, the upper portions of the ridges can be tapered with curved or knife-edge top portions that are shown as flat in FIG. 8. A further possible limitation on the number and thinness of the ridges can be influenced by the removal of the water and C₂-C₄ polyol or hydrophobic coating during the final heat removal step.

In one aspect, the specially fabricated geometry features or ridges are formed of the same material as the target capsule 10. In a further aspect, the ridges are machined features rising from the capsule 10. The ridges are not limited to the same material as the capsule 10.

In a further aspect, the height of the ridges 30, 32 are above the water line. Previous surface treatment for the salt adhesion by machining the base of the capsule 10 to improve salt adhesion resulted in surface roughness finishes below the water line (only 0.1 mm). Having the height of the ridges above the water line increases salt adhesion and heat removal from the salt.

In a further aspect, the above-described hydrophobic coating can be applied to the surfaces of the specially fabricated geometry features or ridges. For example, the hydrophobic coating could by painted or dipped on the ridges. This can further decrease salt creep.

In a further aspect, the above-described final target solution is dispensed into a target capsule 10 with the specially fabricated geometry features or ridges. In still a further aspect, the target capsule 10 and ridges have the hydrophobic coating applied thereto.

The plate 18 of FIG. 4 can be utilized with the target capsule 10 with the specially fabricated geometry features more ridges.

### EXAMPLES

### Example 1

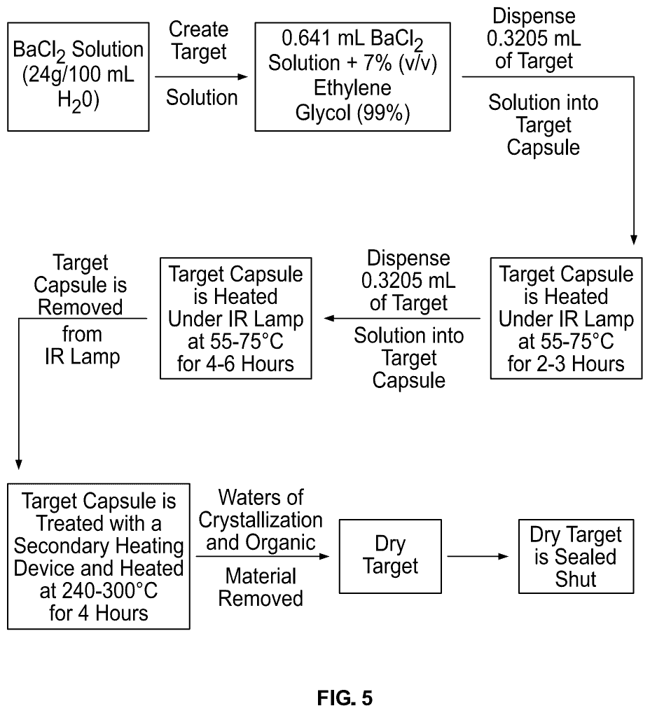

In an illustrative example as shown schematically in FIG. 5, use of 0.641 mL of final target solution including 7% ethylene glycol is added in total using two approximately equal volumes to produce a target that contains 154 mg BaCl₂. If the salt concentration is decreased toward the 50 percent of solubility concentration, a final target solution volume of 0.905 mL would be required that would preferably be dispensed in two volumes of about 0.453 mL each.

The first about one-half volume was heated for about 2 to about 3 hours at about 55 to about 75° C. under the IR lamp, and then permitted to cool (low temperature drying step). The remainder of the target solution was added to the target capsule and the IR heating process was repeated for about 4 to about 6 hours. The target capsule was placed in a secondary heating device and heated at a temperature of about 240 to about 300° C. for 4 hours to produce the dried salt with mitigated salt creep similar to that shown in FIG. 2A.

### Example 2

Surrogate metal salt target studies similar to those of Example 1, those shown in FIGS. 2A and 2B, and schematically shown in FIG. 5 were carried out using Sr(NO₃)₂, CaCl₂, Fe(NO₃)₃, ZnCl₂, Cu(NO₃)₂ as illustrative of other elements treated similarly to the barium salts. Studies were performed with each of these specific salts using the standard parameters utilized for small target barium/radium liquid deposition discussed previously. Some of the studies used ethylene glycol as the creep mitigating agent, whereas others used a hydrophobic coating as the mitigating agent. Each surrogate target salt was also dried without a creep mitigating agent; i.e., as just a water solution to serve as controls.

Thus, where ethylene glycol was the creep mitigating agent, an aqueous solution containing ethylene glycol at 7% (v/v) was prepared to which the surrogate metal salt at a concentration of 12 g/50 mL was added and mixed until dissolution of the metal salt to form a surrogate aqueous target solution.

A surrogate metal salt solution containing only water and the surrogate metal salt at 12 g/50 mL was used as a control for comparison of salt creep to the ethylene glycol-containing solution, for use in studies utilizing a hydrophobic coating, and also a control with no hydrophobic coating.

Two aliquots of roughly equal volume of the study surrogate target salt solutions and their control solutions were added into its own capsule 10 one aliquot at a time, followed by drying and a second aliquot addition of the same study or control solution followed by drying.

Each of the above drying steps utilized infrared (IR) heating 65° C. for approximately 2 hours post each aliquot being delivered into the target capsule; and

Each of the above-dried capsules was then heated to a constant weight by furnace heating at 250° C. for 4 hours.

Thus, each unique material had depositions performed with no mitigation applied (i.e., salt+water), or a potential mitigation agent that was a C₂-C₄ polyol, or a hydrophobic coating applied as described above. It is important to note that no optimization of the parameters to each unique salt form was performed—the optimized parameters of barium/radium deposition were utilized.

The use of a mitigation agent as described herein for each of the alternative surrogate metal salt forms noted provided significant mitigation of salt creep as compared to when no mitigation agent was present. A clear example of this is provided in the Table below, where nearly identical deposit masses of ZnCl₂ were examined-one with mitigation applied and one where no mitigation was present. The mitigation agent in this case was the hydrophobic coating, Aculon® AL-B.

TABLE
ZnCl₂ Deposition & Mitigation of Salt Creep

| Deposit Method | Final Mass [g] | Salt Creep |
|---|---|---|
| Salt + Water | 0.2188 | Significant Salt Creep |
| | | Seen. Splattering of Salt |
| | | During Heating |
| Salt + Water + | 0.2100 | Successful Mitigation |
| Hydrophobic Coating | | |
| Mitigation Agent | | |

Mitigation studies using Sr(NO₃)₂, CaCl₂), Cu(NO₃)₂ and Fe(NO₃)₃ provided similar results to those above. Thus, use of water alone as the solvent resulted in salt creep on drying, whereas utilization of a mitigation agent provided mitigation of the creep. Copper nitrate in the presence of ethylene glycol resulted a bluer color to the solution, the hydrophobic coating was therefore used for the copper nitrate study.

Specific embodiments have been described for the purpose of illustrating the manner in which the invention can be made and used. It should be understood that the implementation of other variations and modifications of this invention and its different aspects will be apparent to one skilled in the art, and that this invention is not limited by the specific embodiments described. Features described in one embodiment can be implemented in other embodiments. The subject disclosure is understood to encompass the present invention and any and all modifications, variations, or equivalents that fall within the spirit and scope of the basic underlying principles disclosed and claimed herein.

Each of the patents, patent applications and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

1. In an aqueous target solution to be dried for the preparation of a metal salt target of a first isotope for heavy ion or electron bombardment and transmutation into a second isotope or a surrogate metal salt for the first isotope that comprises water containing a dissolved target metal salt or a surrogate metal salt therefor present at a concentration of about 25 to about 100 percent by weight of solubility of that metal salt in water at room temperature, the improvement that is the presence of a dissolved C₂-C₄ polyol at about 2 to about 20 percent v/v of the solution, wherein said C₂-C₄ polyol is removable on drying of the target solution, and mitigating salt creep during drying of the target solution.

2. The target solution according to claim 1, wherein said C₂-C₄ polyol has a boiling point at one atmosphere of pressure is about 300° C. or less.

3. The target solution according to claim 1, wherein the first isotope of said dissolved target salt is selected from the group consisting of radium-226, fluorine-19, bromine-79, calcium-48, copper-65, zinc-66, gadolinium-69, molybdenum-100, calcium-44, titanium-48, nickel-58, zinc-68, zirconium-91, barium-131, erbium-167, zinc-69, rubidium-85, nickel-64, strontium-86, ytterbium-176, barium-137.3, copper-63.5, zinc-65.4, strontium-87.6, iron-55.8 and calcium-40.1.

4. A method of preparation of a target for heavy ion or electron irradiation that comprises the steps of:
a) depositing a predetermined first amount of an aqueous liquid target solution containing a dissolved target metal salt including a first isotope for transmutation into a second isotope or a surrogate metal salt for the first isotope by said irradiation, said target metal salt present at a concentration of about 25 to about 100 percent by weight of solubility of that target salt in water at room temperature into a metallic target capsule having an open top; and
i) a dissolved C₂-C₄ polyol present at about 2 to about 20 percent v/v of the solution, wherein said C₂-C₄ polyol has a boiling point at one atmosphere of pressure is about 300° C. or less, or
ii) depositing said aqueous liquid target solution into a target capsule whose wall(s) are coated with a hydrophobic coating to about the level of the top of the deposited first amount of an aqueous liquid target solution, wherein said hydrophobic coating after application and drying, and in a flat horizontal position on the metal of the target capsule exhibits a water contact angle about 70 to about 130°, decomposes at a temperature of about 225° C., and inside bottom of the target capsule is substantially free of said hydrophobic coating;
b) heating the target capsule at a temperature of about 40 to about 100° C. for about 2 to about 6 hours to form a crystalline material;
c) repeating steps a) and b) until the formed crystalline material contains a desired target amount of said target salt; and
d) treating the crystalline material-containing target capsule with a secondary heating source and heating the target capsule at a temperature of about 200 to about 300° C. for a time period of about 4 hours or until the weight of the target capsule when cooled to about room temperature remains constant.

5. The method according to claim 4 including a further step, e) of sealing the target capsule.

6. The method according to claim 4, wherein the first heating of step b) lasts for about 2 to about 3 hours.

7. The method according to claim 6, wherein subsequent heating of steps c) last for about 4 to about 6 hours.

8. The method according to claim 4, wherein said C₂-C₄ polyol is ethylene glycol.

9. The method according to claim 4, further comprising cooling the target capsule with plate in thermal contact with the underside of the target capsule.

10. The method according to claim 9, wherein the plate is made of a high thermal conductivity material.

11. The method according to claim 4, wherein said first isotope of said dissolved target metal salt is selected from the group consisting of radium-226, fluorine-19, bromine-79, calcium-48, copper-65, zinc-66, gadolinium-69, molybdenum-100, calcium-44, titanium-48, nickel-58, zinc-68, zirconium-91, barium-131, erbium-167, zinc-69, rubidium-85, nickel-64, strontium-86, ytterbium-176, barium-137.3, copper-63.5, zinc-65.4, strontium-87.6, iron-55.8 and calcium-40.1.

12. The method according to claim 4, wherein said hydrophobic coating prior to drying contains
(a) 0.1 to 10 percent by weight of a phosphorus acid such as an organo phosphoric acid, an organo phosphinic acid or an phosphonic acid, having perfluorinated hydrocarbon groups capable of forming a self-assembled monolayer on the metal substrate;
(b) 0.1 to 10 percent by weight of a surfactant that is structurally different from (a);
(c) 2 to 30 percent by weight of an organic solvent; and
(d) 50 to 95 percent by weight water;
with the percentages by weight being based on the total weight of (a), (b), (c) and (d).

13. The method according to claim 12, wherein said acid having perfluorinated hydrocarbon groups has the structure: 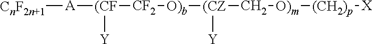
where A is an oxygen radical or a chemical bond;
n is 1 to 20;
Y is H, F, CₙF₂ₙ₊₁, CₙH₂ₙ₊₁;
Z is H or F;
b is 0 to 50;
m is O to 50;
p is 1 to 20; and
X is a group selected from a phosphoric acid, a phosphinic acid and a phosphonic acid.

14. The method according to claim 4, wherein said hydrophobic coating contains reaction product of
(a) a transition metal compound in which the transition metal is selected from niobium and transition metals such as tantalum, titanium, zirconium, lanthanum and tungsten having electrons in the f orbital and in which the transition metal compound has ligands selected from alkoxide, halide, keto acid, amine and acylate, and
(b) a silicon-containing material.

15. The method according to claim 14, wherein said silicon-containing material has a formula selected from:
R¹₄₋ₓSiAₓ or (R¹₃Si)_{y}B or an organo(poly)siloxane and an organo(poly)silazane having units of the formula: 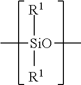
or 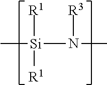
where:
R¹ are identical or different in each presence and are a hydrocarbon or substituted hydrocarbon radical containing from 1 to 100 carbon atoms;
A is hydrogen, halogen, OH, OR² or O—C(O)—R²;
B is NR³_{3-Y};
R² is a hydrocarbon or substituted hydrocarbon radical containing from 1 to 12 carbon atoms;
R³ is hydrogen or is the same as R¹, x is 1, 2 or 3; and y is 1 or 2.

16. A metallic capsule for liquid deposition fabrication of a target for heavy ion or electron bombardment wherein an interior surface of the capsule is coated with a hydrophobic coating, which coating after application, drying and in a flat horizontal position on the metal of the target capsule exhibits a water contact angle about 70 to about 130°, decomposes at a temperature of about 225° C., and inside bottom of the target capsule is substantially free of said hydrophobic coating.

17. A capsule for liquid deposition of a target for heavy ion or electron bombardment wherein the capsule includes one or more ridges extending from an interior bottom of the capsule.

18. The capsule of claim 17, wherein the ridges are formed in a circular shape on the bottom of the capsule.

19. The capsule of claim 17, wherein the ridges are coated with a hydrophobic coating, which coating after application, drying and in a flat horizontal position on the metal of the target capsule exhibits a water contact angle about 70 to about 130°, decomposes at a temperature of about 225° C., and inside bottom of the target capsule is substantially free of said hydrophobic coating.
